(19) United States
(12) Patent Application Publication
(10) Pub. No.: US 2004/0023320 A1
(43) Pub. Date: Feb. 5, 2004

(54) Method and system for analyzing cells

(76) Inventors: Georg E. Steiner, Marchegg (AT); Rupert Ch. Ecker, Vienna (AT)

Correspondence Address: Lerner and Greenberg, P.O. Box 2480, Hollywood, FL, 33020-2480, US

(21) Appl. No.: 10/415,179

(22) PCT Filed: Oct. 23, 2001
(86) PCT No.: PCT/AT01/00343

(30) Foreign Application Priority Data
Oct. 24, 2000 (AT) A 1821/2000

(51) Int. Cl. G06F 19/00; G01N 33/48; G01N 33/50; G01N 1/30; G06K 9/00

(52) U.S. Cl. 435/40.5; 702/19; 382/128

(57) ABSTRACT

The invention relates to a method for analyzing cells that are present as closed clusters. According to said method, a planar tissue preparation is subjected to an identification staining of the cell nuclei and a target structure staining of cell objects that is different from the identification staining. Digital images are recorded of the stained tissue preparation by means of an electronic image recording device and at least one image of a subsection of the tissue cut is displayed in at least one coloration. According to the inventive method, at least one parameter of the cell nuclei and at least one parameter of the cell objects labeled by target structure staining is restricted to a predetermined range of values. Cell nuclei and cell objects whose parameters correspond to the respective parameter range(s) are detected and optionally displayed using image processing algorithms in the image of said subsection. The image content of at least one image detected for the cell nuclei is correlated with the image content of at least one image detected for the target-structure stained cell objects to detect the individual cells. On the basis of the cell nuclei identified a cell growth or a cell enlargement is induced using a predetermined arithmetic algorithm to reconstruct the individual cells. In doing so it is made sure that neighboring cells do not fuse. The number of reconstructed individual cells is determined and/or the individual cells are divided into populations according to certain parameters.

The invention concerns a procedure in accordance with the preamble of the patent claim 1 as well as a set-up according to the preamble of patent claim 12.

The classification of cells in tissue into exactly defined cell types (i.e. epithelial cells, muscle cells, fibroblasts, leukocytes, carcinoma cells, lymphoma) and the categorization of their functional properties are performed using immunohistological methods in cases where morphological characteristics are not sufficient. Highly specific antibodies against characteristic cell type specific antigens are employed in this method. Based on the staining, corresponding conclusions can be drawn.

To date there has been a lack of an objective, automated analysis system that is able to recognize the number of cells found in a specimen, how many of these cells actually react with a specific antibody or antibodies (in the case of multiple staining) and how strong the reaction or the reactions is/are. Currently the standard technique involves visual counting of a representational number of cells and estimation of staining intensity by the examiner. Another option would be to utilize standard image processing software to measure the exact staining intensity that, however, can only be done after manually defining the area of the cell to be measured. If the examiner wants to quantitatively inspect 100 cells and 100 nuclei, she/he has to circle each of the 100 cells and nuclei and only then can she/he perform the measurements. This technique is very time consuming and generally does not give any insight into double, triple, i.e. multiple reactivity. The necessity for a negative and a positive control, which are important for comparison, leads to an exponential increase in workload.

These circumstances are responsible for the fact that results of immunohistological examinations are still not scientifically standardized or comparable and are flawed by subjective bias, problems which have already been overcome in the more recently developed technique of flow cytometry (a comparable system for single cells in suspension). While tetra staining is routinely used in flow cytometry, which allows for exact analysis of percentual distribution and staining intensity of each subpopulation, analysis of immunohistology has not been improved for the last twenty years. To express results from visual examination, mostly methods such as ‘one cross-’ or ‘two cross-positive cells’ are resorted to.

The objective of the invention is the automated recognition of single cells and their components such as nucleus, cytoplasm, and cell membrane in dense, continual tissue structures and the exact measurement of such components without loss of spatial relations to the cells or the ability to identify their localization within the tissue. This should elevate immunohistology to a level of significance similar to that of flow cytometry. In fact, with relation to data comparison with positive and negative controls, classification of cells into single or multiple reactive subpopulations, as well as the quantitative analysis of staining intensities, immunohistology exceeds the capabilities of flow cytometry by far. The wealth of information obtained is greater as spatial relationships can be detected, limited not only to the localization of the cell in tissue but also within the cell itself (membranous, cytoplasmatic or nucleic).

The procedure described at the start, in accordance with the invention, is characterized by claim 1. The set-up described at the start, in accordance with the invention is characterized in claim 12.

It is necessary to use at least one nucleic stain (any regular nucleic stain can be used) and, at least one stain for a target structure, preferably an antibody or antiserum. It is not of vital importance to which cellular structure the antibody or genetic probe or other similar agents bind, as long as the color of the reaction product can be distinguished from the nucleic stain, which is generally the case. There is no limitation as to the amount of stains that can be employed for nuclei and cell structures, as long as distinct physical differentiation of stain color is still possible.

It is also possible to stain cell nuclei as a target structure. In this case nuclei are identity stained at least once and then stained as a target structure. Since images are analyzed separately for each stain it is possible to draw conclusions for the two nucleic stains.

Stains or cell objects as identified in claim 4 are especially relevant as stains or stainable cell components.

In accordance with the invention, for the primary identification of the cell to be analyzed, at least one parameter of the stained nuclei is restricted to a certain value range. This value range should, with the greatest possible probability, encompass a range in which the stained nuclei are found. Objects that are smaller and do not fit into the set range for the parameter “size” should be excluded as these objects do not represent cell nuclei. Larger objects are also filtered out. The number of parameters that can be set to screen for structures that are to be identified as nuclei is not limited. To screen for nuclei or in order to determine parameter ranges it is possible to analyze the relationships of parameters to one another or to look at parameters plotted against each other in form of scattergrams or histograms.

Restriction for the ranges of respective parameters can be performed in accordance with the parameters of the reconstructed single cell. In particular, parameter ranges can be fixed with regard to previously determined analysis results and are fine tuned according to these. Preferably this is done as described in claims 3, 5 and/or 7.

The results of this range restriction, in which parameters are limited to a respective population preferably by using scattergrams and use of a feedback process, lead to an improved object identification strategy that is suited to the properties of the cell nucleus. Furthermore this can be linked to changes in color of the defined population in the original image in order to control the consequences of these operations.

Once identification of the examined portion of the tissue specimen is completed, the defined parameters are used to automatically analyze all further images of original tissue specimen or further tissue specimens from the same or a similar organ.

For analysis of data it is necessary to identify the cell body (cytoplasm) and cell boundary (cell membrane). This requires at least one target structure stain that stains cytoplasm and/or the cell membrane. The characteristic properties for this stain are automatically adjusted using software measurement tools. By marking a stained cell that the examiner chooses from the image, the examiner lays out the region in which measurement of the parameter is to take place. Software measurement tools automatically determine staining intensity, color tone, size and/or shape of the cell. Marking and measuring an unstained cell using the measurement tools can determine boundaries or the difference to unstained cells. This procedure can be performed for every available color channel. Thus it is easily possible to establish surface area covered by a cell. Correlation of image/s stained for target structure and the identity stained image/s makes it possible to examine all objects with comparable properties.

Preferably this is done according to the features described in claim 8. In a cytoplasmatic target structure stain a growth process is initiated starting with the already identified nuclei, which is continued until either a pixel is reached that does not correspond to the selected color tone or the tolerance limit for size and shape (diameter) is exceeded or an object belonging to a neighboring nucleus is reached. Instead of terminating the growth process when reaching an invalid pixel, only that point is not included into the growing binary object. In case of a membrane stain, growth of nuclei is continued up to a point at which an area corresponding to the selected color tone is reached. The process is continued within the stained area until an intensity maximum is reached or a neighboring object or the tolerance limit for size is reached.

In the case of multiple stains that encompass membranous and/or cytoplasmatic elements, both growth processes can be carried out simultaneously and multiple color tones can be used side by side for the purpose of cell reconstruction.

The cells that have been identified in the image using this method are analyzed according to their parameters, preferably staining intensity, size, and shape. The results are presented in form of scattergrams in which they are brought into relation with all of the nuclei in the image. Preferably size is plotted against staining intensity, separately for each color channel. In multiple stains, objects are sorted according to the respective staining intensity and are presented according to the different color channels. In addition it is possible to accentuate identified objects or cells in the original image. Relationships and interdependencies shown in the scattergrams change when parameter settings are altered or newly defined.

Images of all segments of the tissue sample are analyzed in the above-described fashion and the results and derived parameters of single cells are shown in form of scattergrams plotting staining intensities against each other. Analysis for the nucleus, cytoplasm, and cell membrane can be performed separately or in any desired combination. The analysis is preferably performed with intensity scattergrams in which separate populations are graphically discernible and can be separately analyzed. Analysis is performed with respect to the number of examined objects, percentual distribution of the respective objects in different color channels, staining intensity, size and shape. It is possible to limit the analysis to certain populations in the scattergram.

The invention makes possible the precise, automated recognition of individual cells in solid tissues.

The invention is further described in the following figures.

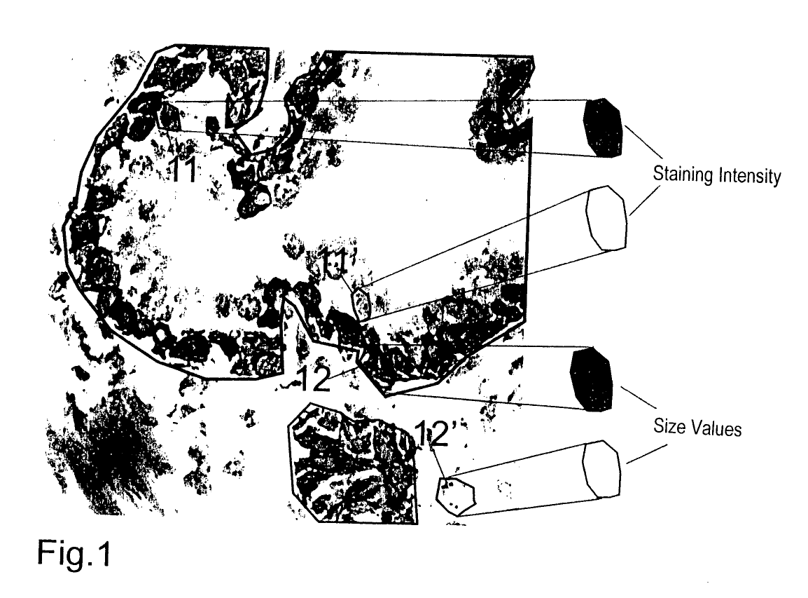

FIG. 1 shows the setting of parameters for cells and setting of a valid parameter range using intuitive software marking tools.

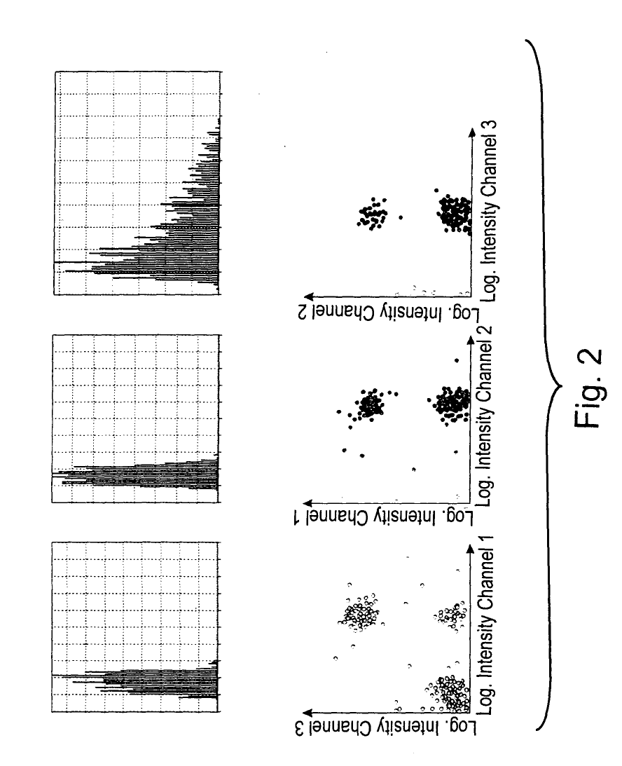

FIG. 2 shows examples of scattergrams and histograms.

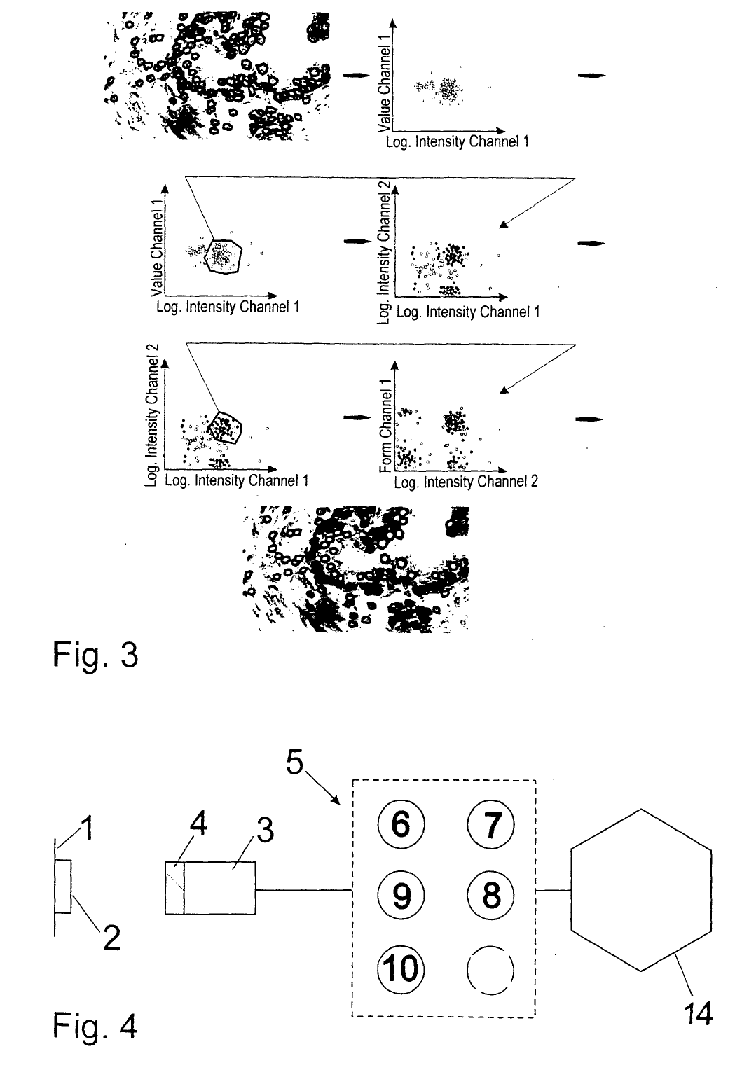

FIG. 3 shows an example for data analysis and

FIG. 4 shows an example of the invention set-up.

FIG. 1 shows a kidney section as an example of a tissue specimen. Cell nuclei (identity stain, in blue) and cytoplasm (target structure stain) were stained. After the staining of tissue specimens parts are defined and scanned, for example, with the help of an Eppendorf micromanipulator. Using a laser-scanning microscope, for example, two scans are performed in the z-plain. One of these is a rough scan, the other a fine scan both of which are guided along a horizontal line in the middle of the image. The focus of the scan can be adjusted to the brightest region. The scan takes, for example, 4 seconds per microscopic field of view, where each section is scanned only once using an argon laser (488 nm). It is possible to undertake multiple scans in sequence. In order to obtain distinctive stains it would be possible, for example, to perform a first scan using 543 nm He/Ne laser so as to measure the fluorochromes cyanine 5 (Cy5) or Cy3. This may be followed by a scan using a 488 nm Argon laser in order to capture the fluorochromes Cy2, fluorescein-isothiocyanat (FITC) or peridinin-chlorophyllprotein (PerCP). Settings of sensitivity of the detectors must be adjusted with both the negative and positive controls and the settings of the parameter regions. The systematic storage of image data of each individual scan consists of up to 8 color channels and is automatically performed.

Software tools that intuitively mark the limits of the respective measurement parameters and also of certain image regions, are employed to adjust the measurement system (FIG. 1). These measurement parameters include size, circumference, shape, staining intensity and color patterns of the examined objects. Restrictions can be defined numerically or graphically. It is preferable to use an intuitive definition for which the user, after studying the image, selects one or more representative measurement objects, i.e. single cells or cellular compartments. The system then extracts from this information, numerical data for the validity interval of every desired parameter. This allows the system to recognize any structures in the images that lie within predefined limitations and correspond to the objects defined as representative.

By marking two nuclei, as is illustrated in FIG. 1, upper panel, the parameter for staining intensity is set as to its minimal and maximal values. These two values represent the limitations for this parameter. All further steps in analysis will only recognize objects as nuclei that have a staining intensity that lies within this fixed range.

To define the parameters for cell size a stain is resorted to that is characteristic for the entire cell, i.e. preferably a membrane stain or cytoplasm stain. These parameters serve as the parameter range for cell size (FIG. 1, lower right panel) Images in which nuclei were defined with respective parameters according to their staining intensity and images in which cell size was defined with respective parameters are correlated. For the purpose of this correlation cell nuclei and cells defined according to their size are matched especially with respect to their location. This type of match can be performed in such a way that a number of different nucleic stains is matched with any one out of a number of different cell stains and vice versa as the correlation of a number of different stains and respective steps of analysis are able to define nuclei, cell size, and entire cells more precisely, which improves accuracy of analysis.

It is possible to mark nuclei and/or cell objects in the image or in a section of a tissue specimen that corresponds to defined or predetermined parameters. Further parameters can be defined and determined for these marked objects, by measuring them. Presentation of the parameters in scattergrams or histograms (FIG. 2) conveys information about the cell population present and can be used to newly define or fine tune earlier parameter ranges so as to further increase precision of analysis.

For the purpose of cell reconstruction or the examination of single cells, cell growth is induced starting from identified nuclei using a predetermined algorithm or one that is adjusted to the tissue sample that is to be examined. A cell area is constructed around cell nuclei, paying particular attention to the maximum and minimum values of cell parameters, especially cell size and cell diameter. It is essential to keep in mind that neighboring, growing cell surfaces do not fuse with each other and that contact of cell surfaces is ruled out. The determined limits of the grown objects are seen as boundaries of the reconstructed solitary cells.

The amount, the area and/or staining intensity related to at least one stain and/or other parameters of the reconstructed solitary cell can be established and/or the solitary cell can be categorized into populations, depending on its staining intensity, or other chosen parameters and can then be presented and further analyzed. The characteristics obtained from the images are available in the form of numerical data and can be depicted and processed in the form of histograms and scattergrams (FIG. 2).

FIG. 2 shows histograms (frequency of staining intensities in color channels) and scattergrams (for single, double and triple reactive cells) for staining intensities in different color channels. Localization and clustering of measurement values of solitary cells can be recognized allowing conclusions to be made about the cells. Scattergrams are especially valuable for further processing of measurement values and for in depth evaluation of numerical data. In these scattergrams various object properties (object shape, surface composition, mean densitometric intensity, fluctuations in intensity, etc) can be plotted against each other thereby showing their interdependence. Such scattergrams make it possible to set gates (validity intervals for any two measurement parameters), which can then be matched with each other (FIG. 3).

FIG. 3 demonstrates the result of identification of the tissue specimen shown in FIG. 1 and the suggested method of processing parameters and image data. A representative, corresponding image with identified cell objects is used to create a scattergram in which object size is plotted against staining intensity (first row of FIG. 3). By the defining of gates, performed either interactively or automatically by determining main clusters of measured points, it is possible to identify specific populations in the measurement data when respective parameters of different color channels are plotted against each other as is shown in row two.

Gates that have been defined in a parameter combination, can also be visualized in other parameter combinations which makes it possible to identify increasingly precise subpopulations as is shown in the third row.

The bottom diagram in FIG. 3 shows that the cell objects shown on the screen 12 can be made identifiable in different ways, i.e. using ungated measurement values, using measurement values in gate 1, using measurement values in gate 1 and 2, using measurement values in gate 1 but not gate 2. The computer can perform identification of the objects, especially using stains, automatically. A prerequisite for this procedure is the presentation of parameters obtained in the correlation process, in form of histograms and scattergrams as well as careful selection of set parameters.

Extremely precise identification of single cells of a certain cell type can be achieved using this method of analysis. Cells encompassed by one gate can be accentuated by color and analyzed separately in other scattergrams in which different measurement parameters are plotted against each other. This makes it possible to examine properties of cells that have already been defined as a population in one gate (i.e. with two measurement parameters) with respect to different object properties without having to reexamine all measured cells in the analysis. Thus the actual evaluation of measured values is not performed through restrictions during the process of image analysis, but rather with regard to certain object properties included in the entirety of measured objects. The parameters obtained in this analysis can be used in order to improve the designation of parameter value ranges.

FIG. 4 shows the schematic representation of the set-up according to the invention. The set up includes a stage 1 for tissue specimens 2. An electronic image recorder 3 through a number of color channels 4 records the respectively stained tissue specimens 2. Images in their respective stain are passed on to a computer 5 in digital form and analyzed by a processor 6. The computer includes a parameter restriction unit 7, preferably a software tool with which value ranges for cell parameters can be defined. This can be performed as is shown in FIG. 1 where a certain nucleus size and stain is selected for nuclei 11, 11′ where a strongly stained 11 and a weakly stained 11′ nucleus are marked. This results in restriction of the parameter staining intensity of nuclei in the image to the minimal and maximal value. Other parameters can also be restricted.

In the same way, the cell size can also be limited to a parameter range. In FIG. 1 (lower panel, right side) a stained, large cell 12 is clearly marked and a negative, smaller cell 12′ is also marked defining the limits for the parameters for cell size and cell color in accordance with the entire extension of the cell or the cell surface. In principle, it is possible to define and set such parameter values and regions manually or according to expected values or according to results obtained in previous analysis. These parameters are then employed in the correlation of identity stained and target structure stained images.

The computer 5 also includes additional memory 8 for the selected parameters and the obtained parameters and additional image memory 9 for the variously stained, recorded digital images.

Correlation of image data acquired for nuclei with image data acquired for target structure stained cell objects is performed in a correlation unit 10. Correlation unit 10 is where calculation of cell growth takes place under special consideration of positioning of identified nuclei and cell surface growth, which extends from these nuclei. Images of stained tissue specimens and/or reconstructed cells and/or histograms and scattergrams are shown on a monitor 12.

Process steps as defined in the invention are performed by computer units i.e. hardware parts and programs in a set-up according to the invention.

What we claim is:

1. A process for the examination, in particular the identification of cells in preferably dense, cohesive cell complexes and solid tissues,
in which a plane tissue sample, especially frozen or paraffin sections, cell smears, cytospin preparations or similar are processed with one or a number of different, especially complete, preferably plane identity stain/s of the cell nucleus, predominantly all nuclei,
in which at least one target structure stain of cell objects, especially of cytoplasm and/or cell membrane and/or cell nucleus and/or further cytological parameter/s of said tissue sample, which differ/s from the identity stain/s, is performed,
in which digital images of said stained tissue specimens are recorded, especially as color or gray tone images, employing an electronic image recorder, for example a laser scanning microscope, a CCD-camera, a video or digital camera, a photo scanner and
in which at least one image of a segment of the said tissue section is represented with at least one stain and/or a selected combination of said stains, said process comprising, in combination:
a restriction of at least one parameter (color tone, surface area, shape, circumference, staining intensity, staining pattern or similar) of said identity stained nuclei in the image of said segment, and at least one parameter (color tone, surface area, shape, circumference, staining intensity, color pattern or similar) of cell objects identified with said target structure stain in an image of said segment, to a selected value range;
application of image processing algorithms to said image of said segment, thereby identifying and showing nuclei and cell objects corresponding to said parameter range/s;
a presentation, if necessary, of measurement results of said parameters of said nuclei and cell objects in said image of said segment obtained with said image processing algorithms in form of histograms and scattergrams in dependency of each other, thereby permitting to set new parameter ranges depending on these measurement results;
a correlation of image data of at least one, preferably all image/s for said cell nuclei with one, preferably all image data for said target structure stained cell objects, in order to determine existent single cells, wherein said correlation is established by employing a predetermined calculation algorithm, starting the cell growth procedure for the reconstruction of single cells from said identified cell nuclei, by, where appropriate, taking into consideration stained areas of stained cell objects identified by at least one target structure stain, with said target structure stain determining at least the cytoplasm and/or the cell membrane of said cell objects;
said cell growth procedure for the reconstruction of single cells, constructing around said cell nuclei a cell area, thereby preferably taking into account maximal and minimal values of said cell parameters, especially of cell size or cell diameter;
said cell growth procedure, paying attention to the criterion, that neighboring cell surfaces do not fuse with each other and that contact of determined cell surfaces is excluded, wherein limitations of said cell objects are used as boundaries for reconstructed single cells, and
where the amount, the area and/or the staining intensity with respect to at least one stain and/or other parameters of the reconstructed single cells are determined and/or single cells are divided into populations with regard to their staining intensity and/or other selected parameters and are further examined, analyzed or shown.

2. A process as claimed in claim 1, further characterized by the fact that images of identified nuclei and/or cell objects can be shown separately or superimposed, i.e. in the same image.

3. A process as claimed in claim 1 or 2, further characterized by the fact that selected parameters, preferably size and/or shape and/or staining intensity of identified nuclei and/or identified cell objects and/or the reconstructed single cells and/or the number of identified nuclei and/or the number of identified cell objects and/or reconstructed single cells can be presented in reciprocate dependency in histograms and/or scattergrams.

4. A process as claimed in one of the claims 1 to 3, further characterized by the use of DNA-stains and/or antibody stains and/or antiserum stains and/or diffusion stains and/or chemical color reactions and/or genetic probe stains, which are employed as identity stains and/or target structure stains, staining cellular objects within the cell or attached to its surface, nuclei, cytoplasm, cell membrane, tumor marker, cytokines, growth factors, ions, specific proteins, DNA sequences or similar.

5. A process as claimed in one of the claims 1 to 4, further characterized by determination of interdependencies of said parameters of nuclei and/or cell objects and/or reconstructed cells, preferably size and/or shape and/or staining intensity, and/or the amount and/or distribution of said nuclei and/or cell objects, and/or by the determination of distribution or population clusters of nuclei and/or cell objects, especially by presentation of said parameter values for reconstructed single cells in scattergrams and/or histograms; said interdependencies being used for
determination of limitations or selection of value ranges for the parameters for the depiction of identity stained nuclei and/or target structure stained cell objects, and
for the realization of cell growth, with
said interdependencies, especially staining intensities in the respective color channels being employed in the assessment of single cells.

6. A process as claimed in one of the claims 1 to 5, wherein after correlation of images of identical tissue specimen segments and reconstruction of single cells for these segments, the predetermined employed parameter values, i.e. ranges can be used in the analysis of images of the remaining segments of the same tissue specimen and/or other tissue specimens.

7. A process as claimed in one of the claims 1 to 6, further characterized by specification and restriction of parameter values for target structure stained cell objects, especially for each existent stain, by marking of depicted cell objects (nuclei, cytoplasm, cell membrane) or any cell object defined as a single cell, before and/or after cell reconstruction,
wherein said specification is achieved by determination of staining intensity, color tone, size and/or shape of the single cell, and
said determination allowing to set new parameter values, i.e. ranges in dependency of said evaluated cell object.

8. A process as claimed in one of the claims 1 to 7, further characterized by a cell reconstruction induced in the form of cell growth starting from the nuclei using the calculation algorithm,
wherein said calculation algorithm continues until the cell membrane reaches a pixel or an object in the image of the tissue specimen,
where the parameter corresponds with the parameter of a cell object or further object that has not been target structure stained in the tissue specimen and/or until a predetermined parameter value is exceeded and/or until the cell growth region of a neighboring nucleus is reached.

9. A process as claimed in one of the claims 1 to 8, further characterized by accentuation or marking of identified single cells, especially in accordance with predetermined parameters and parameter ranges, in the depicted digital image of the tissue specimen, with said image, if necessary, being separated into single color channels corresponding to each stain.

10. A process as claimed in one of the claims 1 to 9, further characterized by
setting of limitations or new definition of parameters, especially of staining intensity and size, prior to picturing of nuclei and cell objects and prior to cell reconstruction,
wherein a depiction and assessment of correlated images with the respective previously determined parameter/s is performed, wherein said parameters for cell reconstruction, predominantly cell size and cell shape, being derived from other parameters of target structure stained cell objects, predominantly surface area, color tone, and staining intensity.

11. A process as claimed in one of the claims 1 to 10, further characterized by the fact that
in a cytoplasmatic stain, a growth process is induced starting from identified nuclei, wherein said growth process continues until either a pixel is reached that does not correspond to the selected color tone or the tolerance limit for size is exceeded or an object belonging to a neighboring nucleus is reached;
in a cell membrane stain, cell growth originating form the nucleus enclosed by the membrane and extending in all directions, terminates where the cell membrane encounters a region or a cell object correspondingly stained to the color of the membrane, or another growing cell object, wherein invasion of the reconstructed cell membrane into the membrane stained region is allowed to a certain degree.

12. A set-up for the examination, in particular the identification of cells in preferably dense, cohesive cell complexes and solid tissues in form of a plane tissue specimen (2), especially in form of frozen or paraffin sections, cell smears, cytospin preparations or similar, with one or a number of different, especially complete, preferably plane identity stain/s of the cell nucleus, predominantly all nuclei, and at least one target structure stain of cell objects, especially of cytoplasm and/or cell membrane and/or cell nucleus and/or further cytological parameter/s that differ/s from the identity stain/s,
wherein said set-up contains an electronic image recorder (3) i.e. laser scanning microscope, CCD-camera, video- or digital camera, photo scanner or similar to record digital images of the stained tissue specimen (2), especially color or gray tone images, attached to which is at least one imaging unit or computer (5) for the production of at least one image of at least one segment of the tissue sample in at least one stain and/or in at least one selectable combination of stains, especially for the processes described in claims 1 to 11,
said set-up comprising, in combination:
a computer (5) for imaging and processing as well as measurement of cell objects in said images, and
a parameter restriction unit (7) with which at least one parameter for identity stained nuclei (11, 11′), for example color tone, surface area, shape, circumference, staining intensity, color pattern or similar and at least one parameter for target structure stained cell objects (12, 12′), for example color tone, surface area, shape, circumference, staining intensity, color pattern or similar can be restricted to a predetermined or selected value range over an input unit, with
said computer (5) being able to identify and accentuate nuclei (11,11′) and cell objects (12,12′) whose parameters correspond with the respective parameter range/s;
a correlation unit (10), in order to determine single cells, with which image data acquired for nuclei from at least one, preferably all image/s are correlated with image data acquired for target structure stained cell objects from at least one, preferably all image/s;
said correlation unit (10), taking into consideration the surface area of target structure stained cell objects determined employing at least one cytoplasm and/or cell membrane target structure stain, and
after which cell growth for the reconstruction of single cells is induced by constructing a cell surface around cell nuclei using a predetermined calculation algorithm,
with said algorithm, predominantly taking into account maximal and minimal values of cell parameters, especially for cell size and circumference with special regard to the criterion that,
neighboring cell surfaces do not fuse and contact of calculated cell surfaces is excluded where the boundary of the determined structure is seen as the boundary for the reconstructed single cell;
wherein the number, surface area and/or staining intensity with regard to at least one stain and/or other parameters of the reconstructed single cell is determined by the computer (5) and/or the single cells are shown with relation to their staining intensity and/or other specified parameters, and/or the data is stored.

13. A set-up according to claim 12, further characterized by an image recorder (3) with multiple color channels (4) for the recording of images of the tissue specimen (2) in different stains.

14. A set-up according to claim 12 or 13, further characterized by the fact that parameters selected with the imaging unit (5), preferably size and/or shape and/or staining intensity, and/or identified nuclei and/or identified cell objects and/or reconstructed single cells and/or the number of identified nuclei and/or the number of identified cell objects and/or reconstructed single cells can be presented in mutual dependency of each other in the form of histograms and/or scattergrams.

15. A set-up according to one of the claims 12 to 14, further characterized by the ability of the imaging unit (5) to accentuate and mark identified single cells in digital images of the tissue specimen, when necessary in separate images for each stain.
